(19) United States
(12) Patent Application Publication
(10) Pub. No.: US 2025/0011441 A1
(43) Pub. Date: Jan. 9, 2025

(54) METHODS OF TREATING CHRONIC GRAFT-VERSUS-HOST DISEASE-RELATED BRONCHIOLITIS OBLITERANS SYNDROME USING AN ANTI-COLONY STIMULATING FACTOR 1 RECEPTOR ANTIBODY

(71) Applicant: Syndax Pharmaceuticals, Inc., Waltham, MA (US)

(72) Inventors: Peter Ordentlich, Lexington, MA (US); Vedran Radojcic, Salt Lake City, UT (US)

(21) Appl. No.: 18/666,375

(22) Filed: May 16, 2024

Related U.S. Application Data
(60) Provisional application No. 63/467,169, filed on May 17, 2023.

(51) Int. Cl. C07K 16/28; A61K 39/00

(52) U.S. Cl. CPC C07K 16/2866; A61K 2039/505; A61K 2039/54; A61K 2039/545

(57) ABSTRACT

The present disclosure provides methods of treating chronic graft-versus-host disease (cGVHD)-related bronchiolitis obliterans syndrome using an antibody that binds to colony stimulating factor 1 receptor (CSF-1R).

### CLAIM OF PRIORITY

This application claims the benefit of U.S. Provisional Patent Application No. 63/467,169, filed on May 17, 2023, which is incorporated by reference herein in its entirety.

### SEQUENCE LISTING

This application contains a Sequence Listing that has been submitted electronically as an XML file named 20443-0821001_SL_ST26.XML. The XML file, created on May 15, 2024, is 23,311 bytes in size. The material in the XML file is hereby incorporated by reference in its entirety.

### BACKGROUND

Chronic graft-versus-host disease (cGVHD) is a severe complication of allogeneic hematopoietic cell transplantation (HCT) that affects various organs leading to a reduced quality of life. cGVHD occurs in up to 50% of allogeneic HCT cases, where donor T- and B-cells derived from the graft recognize and attack host antigens (Socié et al., 2014, Blood, 124:374-84). cGVHD has increased during the last two decades due to increasing patient age and increasing use of unrelated and/or mismatched donors, reduced intensity conditioning regimens, and peripheral blood as source for stem cells (Arai et al., 2015, Biol Blood Marrow Transplant, 21:266-74). Due to an increased risk of non-relapse mortality, cGVHD remains the leading cause for late mortality following allogeneic HCT (Zeiser et al., 2018, Blood, 131:1399-405; Li et al., 2019, Br J Haematol, 184:323-36).

Bronchiolitis obliterans syndrome is a type of obstructive lung disease of the small airways and is one of the most difficult to treat manifestations of cGVHD, with a poor prognosis (Chien et al., 2010, Biol Blood Marrow Transplant, 16:S106-S114). Therapeutic approaches are needed for the treatment of cGVHD-related bronchiolitis obliterans syndrome.

### SUMMARY

The present disclosure provides a method of treating cGVHD-related bronchiolitis obliterans syndrome in a human subject by administering a therapeutically effective amount of an antibody that binds to colony stimulating factor 1 receptor (CSF-1R).

In some embodiments, the antibody comprises a variable heavy (VH) domain comprising VH complementarity determining region (CDR)1 (VHCDR1), VH CDR2, and VH CDR3, wherein: the VH CDR1 comprises the amino acid sequence GFSLTTYGMGVG (SEQ ID NO:6); the VH CDR2 comprises the amino acid sequence NIWWDDDKYYNPSLKN (SEQ ID NO:7); and the VH CDR3 comprises the amino acid sequence IGPIKYPTAPYRYFDF (SEQ ID NO:8); and wherein the antibody comprises a variable light (VL) domain comprising VL CDR1, VL CDR2, and VL CDR3, wherein: the VL CDR1 comprises the amino acid sequence LASEDIYDNLA (SEQ ID NO:9); the VL CDR2 comprises the amino acid sequence YASSLQD (SEQ ID NO: 10); and the VL CDR3 comprises the amino acid sequence LQDSEYPWT (SEQ ID NO:11).

In some embodiments, the VH domain comprises the amino acid sequence EVTLKESGPALVKPTQTLTLTCTFSGFSLTTYGMGVGWIRQPPGKALEWLANIWWD DDKYYNPSLKNRLTISKDTSKNQVVLTMTNMDPVDTATYYCARIGPIKYPTAPYRYF DFWGQGTMVTVS (SEQ ID NO:4) and the VL domain comprises the amino acid sequence DIQMTQSPSSLSASVGDRVTITCLASEDIYDNLAWYQQKPGKAPKLLIYYASSLQDG VPSRFSGSGSGTDYTLTISSLQPEDFATYYCLQDSEYPWTFGGGTKVEIK (SEQ ID NO:5).

In some embodiments, the antibody comprises a heavy chain and a light chain, and wherein the heavy chain comprises the amino acid sequence set forth in SEQ ID NO:12 and the light chain comprises the amino acid sequence set forth in SEQ ID NO:3.

In some embodiments, the antibody is emactuzumab, AMG820, cabiralizumab, IMC-CS4, or DCB-AB21.

In some embodiments, the human subject has received at least two previous cGVHD treatments. In some embodiments, the human subject has received at least three previous cGVHD treatments. In some embodiments, the human subject has received at least four previous cGVHD treatments. In some embodiments, the human subject has received at least five previous cGVHD treatments. In some embodiments, the human subject has received at least six previous cGVHD treatments.

In some embodiments, the antibody is administered intravenously. In some embodiments, the antibody is administered intravenously at a dose of 0.3 mg/kg. In some embodiments, the antibody is administered intravenously at a dose of 1 mg/kg. In some embodiments, the antibody is administered intravenously at a dose of 3 mg/kg. In some embodiments, the antibody is administered intravenously once every two weeks at a dose of 0.3 mg/kg. In some embodiments, the antibody is administered intravenously once every two weeks at a dose of 1 mg/kg. In some embodiments, the antibody is administered intravenously once every four weeks at a dose of 3 mg/kg.

In some embodiments, the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously at a dose of 0.3 mg/kg.

In some embodiments, the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously at a dose of 1 mg/kg.

In some embodiments, the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously at a dose of 3 mg/kg.

In some embodiments, the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously once every two weeks at a dose of 0.3 mg/kg.

In some embodiments, the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously once every two weeks at a dose of 1 mg/kg.

In some embodiments, the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously once every four weeks at a dose of 3 mg/kg.

In some embodiments, the cGVHD is advanced cGVHD.

Other features and advantages of the invention will be apparent from the following detailed description, and from the claims.

### BRIEF DESCRIPTION OF THE DRAWINGS

FIG. 1 is a graph of change from baseline in percent predicted FEV₁ in patients with bronchiolitis obliterans syndrome. Baseline is defined as the latter FEV₁ assessment at screening and cycle 1, day 1 (C1D1) except for 1 subject, whose baseline is the FEV₁ assessment at cycle 1, day 15 (C1D15). Percent change in FEV₁ from baseline is shown for 9 patients; 1 additional patient did not show a progression as reported by change in volume (liters).

### DETAILED DESCRIPTION

The present disclosure provides methods of treating cGVHD-related bronchiolitis obliterans syndrome by administering an antibody that binds to CSF-1R.

CSF-1R is a receptor for the cytokine CSF-1, which is responsible for the production, differentiation, and function of macrophages.

The amino acid sequence of the human CSF-1R protein is:

(SEQ ID NO: 1)
MGPGVLLLLLVATAWHGQGIPVIEPSVPELVVKPGATVTLRCVGNGSVEW
DGPPSPHWTLYSDGSSSILSTNNATFQNTGTYRCTEPGDPLGGSAAIHLY
VKDPARPWNVLAQEVVVFEDQDALLPCLLTDPVLEAGVSLVRVRGRPLMR
HTNYSFSPWHGFTIHRAKFIQSQDYQCSALMGGRKVMSISIRLKVQKVIP
GPPALTLVPAELVRIRGEAAQIVCSASSVDVNFDVFLQHNNTKLAIPQQS
DFHNNRYQKVLTLNLDQVDFQHAGNYSCVASNVQGKHSTSMFFRVVESAY
LNLSSEQNLIQEVTVGEGLNLKVMVEAYPGLQGFNWTYLGPESDHQPEPK
LANATTKDTYRHTFTLSLPRLKPSEAGRYSFLARNPGGWRALTFELTLRY
PPEVSVIWTFINGSGTLLCAASGYPQPNVTWLQCSGHTDRCDEAQVLQVW
DDPYPEVLSQEPFHKVTVQSLLTVETLEHNQTYECRAHNSVGSGSWAFIP
ISAGAHTHPPDEFLFTPVVVACMSIMALLLLLLLLLLYKYKOKPKYQVRW
KIIESYEGNSYTFIDPTQLPYNEKWEFPRNNLQFGKTLGAGAFGKVVEAT
AFGLGKEDAVLKVAVKMLKSTAHADEKEALMSELKIMSHLGQHENIVNLL
GACTHGGPVLVITEYCCYGDLLNFLRRKAEAMLGPSLSPGQDPEGGVDYK
NIHLEKKYVRRDSGFSSQGVDTYVEMRPVSTSSNDSFSEQDLDKEDGRPL
ELRDLLHFSSQVAQGMAFLASKNCIHRDVAARNVLLTNGHVAKIGDFGLA
RDIMNDSNYIVKGNARLPVKWMAPESIFDCVYTVQSDVWSYGILLWEIFS
LGLNPYPGILVNSKFYKLVKDGYQMAQPAFAPKNIYSIMQACWALEPTHR
PTFQQICSFLQEQAQEDRRERDYTNLPSSSRSGGSGSSSSELEEESSSEH
LTCCEQGDIAQPLLQPNNYQFC.

Anti-CSF-1R antibodies can be used to treat cGVHD-related bronchiolitis obliterans syndrome as described herein.

### Anti-Colony Stimulating Factor 1 Receptor Antibodies

### Axatilimab

Axatilimab (also known as SNDX-6352) is a humanized IgG4 monoclonal antibody that binds to CSF-1R and inhibits its function. Axatilimab is described in U.S. Pat. No. 9,908,939, which is incorporated by reference in its entirety.

The amino acid sequences of axatilimab heavy and light chains are shown below. Complementarity-determining regions (CDRs) 1, 2, and 3 of the variable heavy (VH) domain and the variable light (VL) domain are shown in that order from N-terminus to the C-terminus of the mature VL and VH sequences and are both underlined and boldened. Variable regions are underlined. An antibody consisting of the heavy chain (SEQ ID NO:2) and the light chain (SEQ ID NO:3) listed below is termed axatilimab.

### Axatilimab Heavy Chain:

(SEQ ID NO: 2)
EVTLKESGPALVKPTQTLTLTCTFSGFSLTTYGMGVGWIRQPPGKALEWL
ANIWWDDDKYYNPSLKNRLTISKDTSKNQVVLTMTNMDPVDTATYYCARI
GPIKYPTAPYRYFDFWGQGTMVTVSSASTKGPSVFPLAPCSRSTSESTAA
LGCLVKDYFPEPVTVSWNSGALTSGVHTFPAVLQSSGLYSLSSVVTVPSS
SLGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPREE
QENSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLS
LGK.

### Axatilimab Light Chain:

(SEQ ID NO: 3)
DIQMTQSPSSLSASVGDRVTITCLASEDIYDNLAWYQQKPGKAPKLLIYY
ASSLQDGVPSRFSGSGSGTDYTLTISSLQPEDFATYYCLQDSEYPWTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSENRGEC.

The variable heavy (VH) domain of axatilimab has the following amino acid sequence:

(SEQ ID NO: 4)
EVTLKESGPALVKPTQTLTLTCTFSGFSLTTYGMGVGWIRQPPGKALEWL
ANIWWDDDKYYNPSLKNRLTISKDTSKNQVVLTMTNMDPVDTATYYCARI
GPIKYPTAPYRYFDFWGQGTMVTVS.

The variable light (VL) domain of axatilimab has the following amino acid sequence:

(SEQ ID NO: 5)
DIQMTQSPSSLSASVGDRVTITCLASEDIYDNLAWYQQKPGKAPKLLIYY
ASSLQDGVPSRFSGSGSGTDYTLTISSLQPEDFATYYCLQDSEYPWTFGG
GTKVEIK.

The amino acid sequences of the VH CDRs of axatilimab are listed below:

VH CDR1:
(SEQ ID NO: 6)
GFSLTTYGMGVG;
VH CDR2:
(SEQ ID NO: 7)
NIWWDDDKYYNPSLKN;
and
VH CDR3:
(SEQ ID NO: 8)
IGPIKYPTAPYRYFDF.

The amino acid sequences of the VL CDRs of axatilimab are listed below:

VL CDR1:
(SEQ ID NO: 9)
LASEDIYDNLA;
VL CDR2:
(SEQ ID NO: 10)
YASSLQD;
and
VL CDR3:
(SEQ ID NO: 11)
LQDSEYPWT.

### Emactuzumab

Emactuzumab (also known as RG-7155 or R05509554) is a humanized IgG1 monoclonal antibody that binds to CSF-1R and inhibits its function. Emactuzumab is described in U.S. Patent Publication No. 2011-0165156, which is incorporated by reference in its entirety.

The amino acid sequences of emactuzumab heavy and light chains are shown below.

### Emactuzumab Heavy Chain:

(SEQ ID NO: 13)
QVQLVQSGAEVKKPGASVKVSCKASGYTFTSYDISWVRQAPGQGLEWMGV
IWTDGGTNYAQKLQGRVTMTTDTSTSTAYMELRSLRSDDTAVYYCARDQR
LYFDVWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK.

### Emactuzumab Light Chain:

(SEQ ID NO: 14)
DIQMTQSPSSLSASVGDRVTITCRASEDVNTYVSWYQQKPGKAPKLLIYA
ASNRYTGVPSRFSGSGSGTDFTLTISSLQPEDFATYYCQQSFSYPTFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSENRGEC.

### Cabiralizumab

Cabiralizumab (also known as FPA008) is a humanized IgG4 monoclonal antibody that binds to CSF-1R and inhibits its function. Cabiralizumab is described in U.S. Patent Publication No. 2011-0274683, which is incorporated by reference in its entirety.

The amino acid sequences of cabiralizumab heavy and light chains are shown below.

### Cabiralizumab Heavy Chain:

(SEQ ID NO: 15)
QVQLVQSGAEVKKPGSSVKVSCKASGYTFTDNYMIWVRQAPGQGLEWM
GDINPYNGGTTFNQKFKGRVTITADKSTSTAYMELSSLRSEDTAVYYCAR
ESPYFSNLYVMDYWGQGTLVTVSSASTKGPSVFPLAPCSRSTSESTAALG
CLVKDYFPEPVTVSWNSGALTSGVHTFPAVLQSSGLYSLSSWTVPSSSLG
TKTYTCNVDHKPSNTKVDKRVESKYGPPCPPCPAPEFLGGPSVFLFPPKP
KDTLMISRTPEVTCVWDVSQEDPEVQFNWYVDGVEVHNAKTKPREEQENS
TYRWSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVESCSVMHEALHNHYTQKSLSLSLGK.

### Cabiralizumab Light Chain:

(SEQ ID NO: 16)
EIVLTQSPATLSLSPGERATLSCKASQSVDYDGDNYMNWYQQKPGQAPRL
LIYAASNLESGIPARFSGSGSGTDFTLTISSLEPEDFAVYYCHLSNEDLS
TFGGGTKVEIKRTVAAPSVEIFPPSDEQLKSGTASWCLLNNFYPREAKVQ
WKVDNALQSGNSQESVTEQDSKDSTYSLSSTLTLSKADYEKHKVYACEVT
HQGLSSPVTKSENRGEC.

### IMC-CS4

IMC-CS4 (also known as LY3022855) is a humanized IgG1 monoclonal antibody that binds to CSF-1R and inhibits its function. IMC-CS4 is described in U.S. Pat. No. 8,263,079, which is incorporated by reference in its entirety.

The amino acid sequences of IMC-CS4 heavy and light chains are shown below.

### IMC-CS4 Heavy Chain:

(SEQ ID NO: 17)
QDQLVESGGGWQPGRSLRLSCAASGFTESSYGMHWVRQAPGEGLEWVAVI
WYDGSNKYYADSVKGRFTISRDNSKNTLYLQMNSLRAEDTAVYYCARGDY
EVDYGMDVWGQGTTVTVASASTKGPSVFPLAPSSKSTSGGTAALGCLVKD
YFPEPVTVSWNSGALTSGVHTFPAVLQSSGLYSLSSWTVPSSSLGTQTYI
CNVNHKPSNTKVDKRVEPKSCDKTHTCPPCPAPELLGGPSVFLFPPKPKD
TLMISRTPEVTCVWDVSHEDPEVKENWYVDGVEVHNAKTKPREEQYNSTY
RWSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK.

### IMC-CS4 Light Chain:

(SEQ ID NO: 18)
AIQLTQSPSSLSASVGDRVTITCRASQGISNALAWYQQKPGKAPKLLIYD
ASSLESGVPSRFSGSGSGTDFTLTISSLQPEDFATYYCQQFNSYPWTFGQ
GTKVEIKRTVAAPSVFIFPPSDEQLKSGTASWCLLNNFYPREAKVQWKVD
NALQSGNSQESVTEQDSKDSTYSLSSTLTLSKADYEKHKVYACEVTHQGL
SSPVTKSENRGEC.

### AMG820

AMG820 (also known as AMB-05X) is a humanized IgG2 monoclonal antibody that binds to CSF-1R and inhibits its function. AMG820 is described in International Application No. 2009-026303, which is incorporated by reference in its entirety.

The amino acid sequences of AMG820 heavy and light chains are shown below.

### AMG820 Heavy Chain:

(SEQ ID NO: 19)
QVQLVQSGAEVKKPGASVKVSCKASGYTFTSYGISWVRQAPGQGLEWMGW
ISAYNGNTNYAQKLQGRVTMTTDTSTSTAYMELRSLRSDDTAVYYCARES
WFGEVFFDYWGQGTLVTVSSASTKGPSVFPLAPCSRSTSESTAALGCLVK
DYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTL
MISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTL
PPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK.

### AMG820 Light Chain:

(SEQ ID NO: 20)
DIVMTQSPDSLAVSLGERATINCKSSQSVLDSSDNKNYLAWYQQKPGQPP
KLLIYWASNRESGVPDRESGSGSGTDFTLTISSLQAEDVAVYYCQQYYSD
PFTFGPGTKVDIKRTVAAPSVFIFPPSDEQLKSGTASVVVCLLNNFYPRE
AKVQWKVDNALQSGNSQESVTEQDSKDSTYSLSSTLTLSKADYEKHKVYA
CEVTHQGLSSPVTKSENRGEC.

### DCB-AB21

DCB-AB21 is a humanized monoclonal antibody that binds to CSF-1R and inhibits its function. DCB-AB21 is described in U.S. Patent Application No. 2022-0064310, which is incorporated by reference in its entirety.

In certain embodiments, an anti-CSF-1R antibody described herein includes a human heavy chain and light chain constant region. In certain embodiments, the heavy chain constant region comprises a CH1 domain and a hinge region. In some embodiments, the heavy chain constant region comprises a CH2 domain. In some embodiments, the heavy chain constant region comprises a CH3 domain. In some embodiments, the heavy chain constant region comprises CH1, CH2 and CH3 domains. If the heavy chain constant region includes substitutions, such substitutions can modify the properties of the antibody (e.g., increase or decrease one or more of: Fc receptor binding, antibody glycosylation, the number of cysteine residues, effector cell function, or complement function). In certain embodiments, the antibody is an IgG antibody. In specific embodiments, the antibody is selected from the group consisting of IgG1, IgG2, IgG3, and IgG4.

In some embodiments, an anti-CSF-1R antibody is provided wherein a C-terminal residue of an antibody sequence described herein is cleaved, for example, the C-terminal residue of a heavy chain sequence, for example, a terminal lysine. Generally, the cleavage results from post-translation modifications of the expressed antibody. For example, an anti-CSF-1R antibody can include a heavy chain comprising the amino acid sequence set forth in SEQ ID NO:12 (below) and a light chain comprising the amino acid sequence set forth in SEQ ID NO:3.

(SEQ ID NO: 12)
EVTLKESGPALVKPTQTLTLTCTFSGFSLTTYGMGVGWIRQPPGKALE
WLANIWWDDDKYYNPSLKNRLTISKDTSKNQVVLTMTNMDPVDTATYYCA
RIGPIKYPTAPYRYFDFWGQGTMVTVSSASTKGPSVFPLAPCSRSTSEST
AALGCLVKDYFPEPVTVSWNSGALTSGVHTFPAVLQSSGLYSLSSVVTVP
SSSLGTKTYTCNVDHKPSNTKVDKRVESKYGPPCPPCPAPEFLGGPSVEL
FPPKPKDTLMISRTPEVTCVVVDVSQEDPEVQFNWYVDGVEVHNAKTKPR
EEQENSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLS
LSLG.

Antibodies can be made, for example, by preparing and expressing synthetic genes that encode the recited amino acid sequences or by mutating human germline genes to provide a gene that encodes the recited amino acid sequences. Moreover, this antibody and other anti-CSF-1R antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., Bio Techniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J Mol. Biol., 227:776-798 (1992); Cook, G. P. et al., Immunol. Today, 16: 237-242 (1995); Chothia, D. et al., J Mol. Bio. 227:799-817 (1992); and Tomlinson et al., EMBO J, 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

The anti-CSF-1R antibodies can be in the form of full length antibodies, or in the form of low molecular weight forms (e.g., biologically active antibody fragments or minibodies) of the anti-CSF-1R antibodies, e.g., Fab, Fab′, F(ab′)₂, Fv, Fd, dAb, scFv, and sc(Fv)2. Other anti-CSF-1R antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an anti-CSF-1R antibody and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-CSF-1R antibody comprising at least one deamidation site, wherein the pH of the composition is from about 5.0 to about 6.5, such that, e.g., at least about 90% of the anti-CSF-1R antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.0, 5.1, 5.2, 5.3, 5.4, 5.5, 5.6, 5.7, 5.8, 5.9, 6.0, 6.1, 6.2, 6.3, 6.4 or 6.5.

An “acidic variant” is a variant of a polypeptide of interest which is more acidic (e.g., as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e., the neutral amide side chain has been converted to a residue with an overall acidic character.

The term “mixture” as used herein in reference to a composition comprising an anti-CSF-1R antibody means the presence of both the desired anti-CSF-1R antibody and one or more acidic variants thereof. The acidic variants may comprise predominantly deamidated anti-CSF-1R antibody, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

### Bispecific Antibodies

In certain embodiments, an anti-CSF-1R antibody described herein is present in a bispecific antibody. Bispecific antibodies are antibodies that have binding specificities for at least two different epitopes. Exemplary bispecific antibodies may bind to two different epitopes of the CSF-1R protein. Other such antibodies may combine a CSF-1R binding site with a binding site for another protein. Bispecific antibodies can be prepared as full length antibodies or low molecular weight forms thereof (e.g., F(ab′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

In certain embodiments, an anti-CSF-1R antibody thereof described herein is present in a multivalent antibody. A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

The antibodies disclosed herein may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g., ⁹⁰Y, ¹³¹I) fluorescent substances, luminescent substances, haptens, enzymes, metal chelates, drugs, and toxins (e.g., calicheamicin, Pseudomonas exotoxin A, ricin (e.g., deglycosylated ricin A chain), exatecan, auristatins (e.g., auristatin E), maytansine, pyrrolobenzodiazepine (PBD)).

In one embodiment, to improve the cytotoxic actions of anti-CSF-1R antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-CSF-1R antibody is modified with a moiety that improves its stabilization and/or retention in circulation, e.g., in blood, serum, or other tissues, e.g., by at least about 1.5, 2, 5, 10, or 50 fold. For example, the anti-CSF-1R antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-CSF-1R antibody can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 5,156,840).

### Methods of Producing Antibodies

Antibodies may be produced in, for example, bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Nat. Acad. Sci. USA 77:4216-4220, used with a DHFR selectable marker, e.g., as described in Kaufman and Sharp (1982)Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-CSF-1R antibody (e.g., axatilimab) is introduced into dhfr CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Antibody Pharmaceutical Compositions and Administration

An anti-CSF-1R antibody described herein can be formulated as a pharmaceutical composition for administration to a human subject, e.g., to treat a disorder described herein. Typically, a pharmaceutical composition includes a pharmaceutically acceptable carrier. As used herein, pharmaceutically acceptable carrier includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X). Suitable anti-CSF-1R antibody formulations are described, e.g., in U.S. Pat. No. 10,039,826 B2, which is incorporated by reference in its entirety.

The anti-CSF-1R antibody can be administered to a human subject having cGVHD-related bronchiolitis obliterans syndrome. In some embodiments, the anti-CSF-1R antibody can be administered to a human subject having advanced cGVHD-related bronchiolitis obliterans syndrome.

The anti-CSF-1R antibody can be administered to a human subject having cGVHD-related bronchiolitis obliterans syndrome who has received one or more previous cGVHD treatments. In some embodiments, the human subject has received at least two previous cGVHD treatments. In some embodiments, the human subject has received at least three previous cGVHD treatments. In some embodiments, the human subject has received at least four previous cGVHD treatments. In some embodiments, the human subject has received at least five previous cGVHD treatments. In some embodiments, the human subject has received at least six previous cGVHD treatments.

The anti-CSF-1R antibody can be administered to a human subject, e.g., a human subject in need thereof, for example, by a variety of methods. For many applications, the route of administration is one of intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody can also be tailored for the individual case, e.g., by monitoring the human subject, e.g., using tomographic imaging, e.g., to visualize the lung.

The antibody can be administered as a fixed dose, or in a mg/kg subject weight dose (as used herein, “mg/kg” refers to mg of an antibody administered per kg of body weight of the treated subject). The dose can also be chosen to reduce or avoid production of antibodies against the anti-CSF-1R antibody. Dosage regimens are adjusted to provide the desired response, e.g., a therapeutic response. Generally, doses of the anti-CSF-1R antibody can be used in order to provide a human subject with the agent in bioavailable quantities. For example, doses in the range of about 0.1 mg/kg to about 30 mg/kg can be administered. In specific embodiments, a human subject is administered the antibody at a dose of about 0.1 mg/kg to about 10 mg/kg (e.g., a dose of about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.5 mg/kg, 2 mg/kg, 2.5 mg/kg, 3 mg/kg, 4 mg/kg, 5 mg/kg, 6 mg/kg, 7.5 mg/kg, or about 10 mg/kg). In other embodiments, a human subject is administered the antibody at a dose of about 0.3 mg/kg to about 3 mg/kg (e.g., a dose of about 0.3 mg/kg, 1 mg/kg, or 3 mg/kg). As used herein, “about” when referring to a measurable value such as an amount, a dosage, a temporal duration, and the like, is meant to encompass variations of 10%. For example, with respect to doses or dosages, the term “about” denotes a range that is ±10% of a recited dose, such that, for example, a dose of about 3 mg/kg will be between 2.7 mg/kg and 3.3 mg/kg subject weight. In another example, exemplary doses included within “about 0.3 mg/kg” are 0.27 mg/kg, 0.28 mg/kg, 0.29 mg/kg, 0.3 mg/kg, 0.31 mg/kg, 0.32 mg/kg, and 0.33 mg/kg.

Dosage unit form or “fixed dose” or “flat dose” as used herein refers to physically discrete units suited as unitary dosages for the human subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion.

An anti-CSF-1R antibody dose can be administered, e.g., at a periodic interval over a period of time (a course of treatment) sufficient to encompass at least 2 doses, 3 doses, 5 doses, 10 doses, or more, e.g., once or twice daily, or about one to four times per week (e.g., at least twice per week), or weekly, biweekly (every two weeks), every three weeks, every four weeks, monthly, e.g., for between about 1 to 12 weeks. Factors that may influence the dosage and timing required to effectively treat a human subject, include, e.g., the severity of the disease or disorder, formulation, route of delivery, previous treatments, the general health and/or age of the human subject, and other diseases present. Moreover, treatment of a human subject with a therapeutically effective amount of a compound can include a single treatment or, preferably, can include a series of treatments.

An exemplary weight-based dosing regimen comprises intravenous administration of an anti-CSF-1R antibody (e.g., axatilimab) at a dosage of about 10.3 mg/kg once every two weeks.

A further exemplary weight-based dosing regimen comprises intravenous administration of an anti-CSF-1R antibody (e.g., axatilimab) at a dosage of about 1 mg/kg once every two weeks.

A further exemplary weight-based dosing regimen comprises intravenous administration of an anti-CSF-1R antibody (e.g., axatilimab) at a dosage of about 3 mg/kg once every four weeks.

A pharmaceutical composition may include a therapeutically effective amount of an anti-CSF-1R antibody described herein. The term “therapeutically effective amount of an agent is that amount sufficient to effect beneficial or desired results, for example, clinical results, and, as such, a “therapeutically effective amount” depends upon the context in which it is being applied. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### Indications

An anti-CSF-1R antibody described herein (e.g., axatilimab) can be used to treat chronic graft-versus-host disease (cGVHD)-related bronchiolitis obliterans syndrome. In some embodiments, the treated subject has advanced cGVHD. In some embodiments, the treated subject has received at least two previous cGVHD treatments. In some embodiments, the treated subject has received at least three previous cGVHD treatments. In some embodiments, the treated subject has received at least four previous cGVHD treatments. In some embodiments, the treated subject has received at least five previous cGVHD treatments. In some embodiments, the treated subject has received at least six previous cGVHD treatments.

Another aspect comprises an anti-CSF-1R antibody described herein (e.g., axatilimab) for use in the treatment of cGVHD-related bronchiolitis obliterans syndrome. In some embodiments, the treated subject has advanced cGVHD. In some embodiments, the treated subject has received at least two previous cGVHD treatments. In some embodiments, the treated subject has received at least three previous cGVHD treatments. In some embodiments, the treated subject has received at least four previous cGVHD treatments. In some embodiments, the treated subject has received at least five previous cGVHD treatments. In some embodiments, the treated subject has received at least six previous cGVHD treatments.

Another aspect comprises an anti-CSF-1R antibody described herein (e.g., axatilimab) in the manufacture of a medicament for treating cGVHD-related bronchiolitis obliterans syndrome. In some embodiments, the treated subject has advanced cGVHD. In some embodiments, the treated subject has received at least two previous cGVHD treatments. In some embodiments, the treated subject has received at least three previous cGVHD treatments. In some embodiments, the treated subject has received at least four previous cGVHD treatments. In some embodiments, the treated subject has received at least five previous cGVHD treatments. In some embodiments, the treated subject has received at least six previous cGVHD treatments.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Safety, Tolerability, and Efficacy of Axatilimab in Advanced Chronic Graft-Versus-Host Disease-Related Bronchiolitis Obliterans Syndrome (BOS)

Safety and efficacy of axatilimab was assessed in a phase 1/2, open-label, dose-escalation study in participants with advanced chronic graft versus host disease (cGVHD).

In this study, 32 patients were treated at 2 different doses (1 mg/kg every 2 weeks [n=26] and 3 mg/kg every 4 weeks [n=6]).

The BOS definition used in this study followed the National Institutes of Health (NIH) cGVHD criteria (Jagasia et al., Biol Blood Marrow Transplant 2015; 21:389-401):
Obstructive lung defect with forced expiratory volume in 1 second (FEV₁) <75% predicted;
FEV₁/vital capacity <0.7;
Evidence of air trapping; and
Absence of infection.

BOS response was defined by the NIH consensus criteria as either (Lee et al. Biol Blood Marrow Transplant 2015; 21:984-999):
≥10% absolute improvement in percent predicted FEV₁; and
Lung symptom score improvement by 1 point.

BOS response and time to first BOS response were evaluated. Safety outcomes were also reported.

Patient Baseline Demographics: BOS was present in 15 of 32 patients included in the study (Table 1). Patients with BOS had more heavily pretreated cGVHD at study entry compared with the overall patient population. Patients with BOS received a median of 5 (range, 2 to 11) previous cGVHD treatments compared with a median of 3.5 (range, 2 to 11) previous treatments in the full cohort.

TABLE 1
Patient Demographics and Pretreatment Characteristics

| | Full Cohort | | Patient with BOS | |
|---|---|---|---|---|
| | N = 32 | | n = 15 | |
| | Axatilimab | Axatilimab | Axatilimab | Axatilimab |
| | 1 mg/kg q2w | 3 mg/kg q4w | 1 mg/kg q2w | 3 mg/kg q4w |
| Patient Characteristics | n = 26 | n = 6 | n = 11 | n = 4 |
| Age, mean (SD) | 51.5 (14.6) | 59.2 (15.3) | 47.1 (14.7) | 63.3 (7.5) |
| Male, n (%) | 16 (61.5) | 4 (66.7) | 6 (54.5) | 2 (50.0) |
| Time from cGVHD | 3.3 (0.35, | 1.6 (0.75, | 3.8 (1.86, | 2.6 (1.23, |
| diagnosis to study start, | 7.10)^{a} | 3.91)^{b} | 7.10) | 3.91) |
| years, median (range) | | | | |
| Previous GVHD | 3.5 (2, | 3.5 (2, | 6.0 (3, | 3.5 (2, |
| treatments, median | 11) | 9) | 11) | 9) |
| (range) | | | | |

BOS, bronchiolitis obliterans syndrome; cGVHD, chronic graft-versus-host disease; q2w, once every 2 weeks; q4w, once every 4 weeks.
^{a}n = 25.
^{b}n = 5.

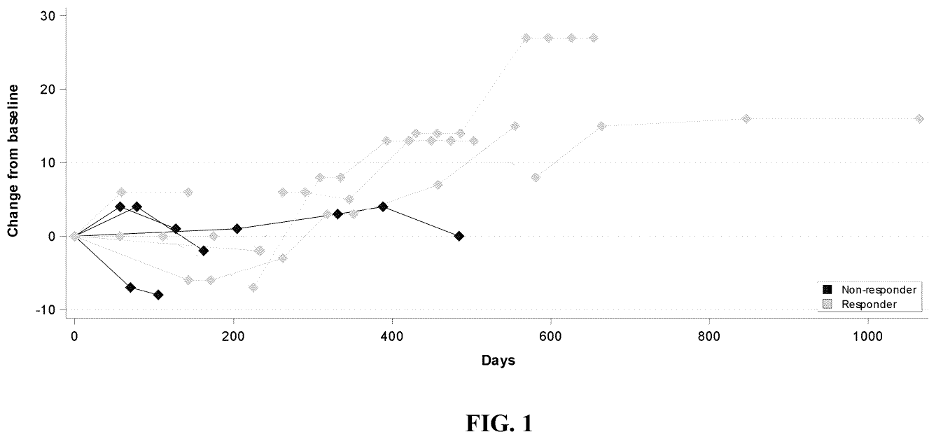

BOS Response: 8 of 15 patients with BOS demonstrated partial response, with best absolute improvement in FEV₁≥10% seen in 3 patients and symptom-only improvement in 5 patients (Table 2). In all patients with BOS, time to first BOS response (symptom or FEV₁ improvement) was 2.76 months (range 0.95-18.23); segmentation according to dosage is reported in Table 2. None of 15 patients with BOS experienced progression, including 10 patients in whom post-baseline spirometry results were available (FEV₁ monitoring not mandated by protocol; FIG. 1). These results demonstrated that most patients with BOS had improvement in FEV₁ without disease progression.

TABLE 2
BOS response and Time to BOS Response

Patients with BOS
n = 15

| | Axatilimab | Axatilimab |
|---|---|---|
| | 1 mg/kg q2w | 3 mg/kg q4w |
| | n = 11 | n = 4 |
| BOS response, n | | |
| Total | 5 | 3 |
| FEV₁ (% predicted) | 2 | 1 |
| Symptom | 4 | 3 |
| Symptom only | 3 | 2 |
| Time to BOS response | | |
| Months, median (range) | 2.76 (0.95, 18.23) | 2.79 (1.94, 2.83) |

BOS, bronchiolitis obliterans syndrome; FEV₁, forced expiratory volume in 1 second; q2w, once every 2 weeks; q4w, once every 4 weeks.

Treatment Duration, Discontinuations, and Adverse Events: Median time on study treatment was 6.9 months. Few patients discontinued treatment owing to adverse events (AEs) or interrupted treatment for any reason (Table 3).

TABLE 3
Axatilimab Dose Changes and Treatment Parameters
in the Full Study Cohort and Patients With BOS

| | Axatilimab | Axatilimab | Patients |
|---|---|---|---|
| | 1 mg/kg q2w | 3 mg/kg q4w | with BOS |
| | n = 26 | n = 6 | n = 15 |
| Axatilimab dose changes | | | |
| due to AE,^{a} n(%) | | | |
| Discontinuation | 2 (7.7) | 1 (16.7) | 1 (6.7) |
| Dose decrease | 2 (7.7) | 1 (16.7) | 2 (13.3) |
| Axatilimab treatment | | | |
| Axatilimab cycles started, | 7.5 (1, | 7.5 (3, | 7.0 (2, |
| median (range) | 39) | 24) | 39) |
| Axatilimab treatment | 7.13 (0.95, | 7.70 (2.79, | 6.93 (1.41, |
| duration, months, | 40.28) | 27.86) | 40.28) |
| median (range) | | | |

AE, adverse event; cGVHD, chronic graft-versus-host disease.
^{a}Except AE of cGVHD disease progression.

In the BOS cGVHD population, adverse events (AEs) related to axatilimab occurred in 11 patients, with 2 patients experiencing ≥grade 3 AEs (Table 4). On-target effects of CSF-1R blockade related to tissue macrophage depletion, Kupffer cells included, were seen in reversible periorbital edema and transient enzyme elevations (alanine and aspartate aminotransferase, creatinine phosphokinase, amylase, lipase) without evidence of concurrent de novo end organ toxicity. Infectious risk was comparable to that reported for cGVHD patients treated with other FDA-approved agents.

TABLE 4
AEs Related to Axatilimab Treatment in the
Full Study Cohort and Patients With BOS

| | Axatilimab | Axatilimab | Patients |
|---|---|---|---|
| | 1 mg/kg q2w | 3 mg/kg q4w | with BOS |
| All related terms in ≥10% | n = 26 | n = 6 | n = 15 |
| Related TEAE, n(%) | 19 (73.1) | 5 (83.3) | 11 (73.3) |
| AST increase | 6 (23.1) | 3 (50.0) | 4 (26.7) |
| Fatigue | 6 (23.1) | 2 (33.3) | 3 (20.0) |
| CPK increase | 3 (11.5) | 4 (66.7) | 5 (33.3) |
| ALT increase | 3 (11.5) | 2 (33.3) | 2 (13.3) |
| Amylase increase | 4 (15.4) | 0 | 1 (6.7) |
| Lipase increased | 3 (11.5) | 3 (50.0) | 2 (13.3) |
| Periorbital edema | 3 (11.5) | 3 (50.0) | 5 (33.3) |
| Lactate dehydrogenase | 1 (3.8) | 2 (33.3) | 3 (20.0) |
| increased | | | |
| Nausea | 3 (11.5) | 1 (16.7) | 3 (20.0) |
| At least 1 related | 4 (15.4) | 2 (33.3) | 2 (13.3) |
| grade ≥3 TEAE, n (%) | | | |
| Infection and infestations | 5 (19.2) | 1 (16.7) | 2 (13.3) |

ALT, alanine aminotransferase; AST, aspartate aminotransferase; CPK, blood creatine kinase; q2w, once every 2 weeks; q4w, once every 4 weeks; TEAE, treatment-emergent adverse event.

Taken together, the results described herein demonstrate that axatilimab has clinical activity against BOS and that axatilimab has a manageable safety profile. Adverse events were driven by a CSF-1R blockade effect, which indicates on-target macrophage depletion.

### Example 2: Efficacy and Safety of Axatilimab at Three Different Doses in Patients with Advanced Chronic Graft-Versus-Host Disease-Related Bronchiolitis Obliterans Syndrome (BOS)

Efficacy and safety of axatilimab was assessed in a phase 2, open-label, randomized, multicenter study of axatilimab in patients with recurrent/refractory cGVHD randomized patients 1:1:1 to intravenous axatilimab at 0.3 mg/kg once every 2 weeks (Q2W, n=80), 1 mg/kg Q2W (n=81), or 3 mg/kg once every 4 weeks (Q4W, n=80). BOS and cGVHD diagnosis and response criteria followed 2014 National Institutes of Health cGVHD Consensus. The primary efficacy endpoint was cGVHD overall response rate (ORR) in the first 6 cycles (24 weeks). Safety endpoints included frequency and severity of treatment-emergent adverse events (TEAEs).

BOS was present in 108 (45%) patients (Table 5). The median time to BOS response was <3 months. BOS response rate was highest at the 0.3 mg/kg dose. The primary ORR endpoint was met in all cohorts. As shown in Table 5, patients treated with axatilimab at doses of 0.3 mg/kg once every two weeks, 1.0 mg/kg once every two weeks, and 3.0 mg/kg once every four weeks demonstrated overall response rates within the first six cycles of treatment of 74%, 67%, and 50%, respectively. TEAEs were dose dependent in frequency and severity, with majority on-target effects of CSF-1R blockade. TEAE-driven discontinuation occurred in 6% of all patients in the 0.3 mg/kg cohort and 16% of all study patients.

TABLE 5
Patient baseline and response characteristics

| | 0.3 mg/kg | 1 mg/kg | 3 mg/kg |
|---|---|---|---|
| | Q2W | Q2W | Q4W |
| Characteristic (%) | n = 80 | n = 81 | n = 80 |
| Baseline BOS | 40 | 51 | 44 |
| FEV₁ ≤ 39% | 47 | 42 | 26 |
| ORR | 74 | 67 | 50 |
| BOS response | 47 | 34 | 37 |

BOS, bronchiolitis obliterans syndrome; FEV₁, forced expiratory volume in 1 second; q2w, once every 2 weeks; q4w, once every 4 weeks.

Thus, these results demonstrate a durable patient response to treatment with axatilimab, even at the lowest dose of 0.3 mg/kg once every two weeks. These results also demonstrate that treatment with axatilimab was well tolerated by patients. Axatilimab at the lowest dose tested of 0.3 mg/kg once every two weeks showed the best efficacy and safety in cGVHD and BOS.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating chronic graft-versus-host disease (cGVHD)-related bronchiolitis obliterans syndrome in a human subject in need thereof, the method comprising administering to the human subject a therapeutically effective amount of an antibody that binds to colony stimulating factor 1 receptor (CSF-1R).

2. The method of claim 1, wherein the antibody comprises a variable heavy (VH) domain comprising VH complementarity determining region (CDR)1 (VHCDR1), VH CDR2, and VH CDR3, wherein:
the VH CDR1 comprises the amino acid sequence GFSLTTYGMGVG (SEQ ID NO:6);
the VH CDR2 comprises the amino acid sequence NIWWDDDKYYNPSLKN (SEQ ID NO:7); and
the VH CDR3 comprises the amino acid sequence IGPIKYPTAPYRYFDF (SEQ ID NO:8); and
wherein the antibody comprises a variable light (VL) domain comprising VL CDR1, VL CDR2, and VL CDR3, wherein:
the VL CDR1 comprises the amino acid sequence LASEDIYDNLA (SEQ ID NO:9);
the VL CDR2 comprises the amino acid sequence YASSLQD (SEQ ID NO: 10); and
the VL CDR3 comprises the amino acid sequence LQDSEYPWT (SEQ ID NO:11).

3. The method of claim 2, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:4) and the VL domain comprises the amino acid sequence DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:5).

4. The method of claim 1, wherein the antibody comprises a heavy chain and a light chain, and wherein the heavy chain comprises the amino acid sequence set forth in SEQ ID NO:12 and the light chain comprises the amino acid sequence set forth in SEQ ID NO:3.

5. The method of claim 1, wherein the antibody is emactuzumab, AMG820, cabiralizumab, IMC-CS4, or DCB-AB21.

6. The method of claim 1, wherein the human subject has received at least two, at least three, at least four, at least five, or at least six previous cGVHD treatments.

7.-10. (canceled)

11. The method of claim 1, wherein the antibody is administered intravenously.

12. The method of claim 1, wherein the antibody is administered intravenously at a dose of 0.3 mg/kg.

13. The method of claim 1, wherein the antibody is administered intravenously at a dose of 1 mg/kg.

14. The method of claim 1, wherein the antibody is administered intravenously at a dose of 3 mg/kg.

15. The method of a claim 1, wherein the antibody is administered intravenously once every two weeks at a dose of 0.3 mg/kg.

16. The method of claim 1, wherein the antibody is administered intravenously once every two weeks at a dose of 1 mg/kg.

17. The method of claim 1, wherein the antibody is administered intravenously once every four weeks at a dose of 3 mg/kg.

18. The method of at claim 1, wherein the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously at a dose of 0.3 mg/kg.

19. The method of claim 1, wherein the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously at a dose of 1 mg/kg.

20. The method of claim 1, wherein the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously at a dose of 3 mg/kg.

21. The method of claim 1, wherein the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously once every two weeks at a dose of 0.3 mg/kg.

22. The method of claim 1, wherein the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously once every two weeks at a dose of 1 mg/kg.

23. The method of at claim 1, wherein the antibody comprises a heavy chain and a light chain, wherein the heavy chain comprises the amino acid sequence set forth in SEQ ID NO:12 and the light chain comprises the amino acid sequence set forth in SEQ ID NO:3, and wherein the antibody is administered intravenously once every four weeks at a dose of 3 mg/kg.

24. The method of claim 1, wherein the cGVHD is advanced cGVHD.


SEQUENCE LISTING (20 sequences)

SEQ ID NO: 1 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 2 - protein, 453 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 214 aa, synthetic construct
DIQMTQSPSSLSASVGDRVTITCLASEDIYDNLAWYQQKPGKAPKLLIYYASSLQDGVPSRFSGSGSGTDYTLTISSLQPEDFATYYCLQDSEYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 125 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVS

SEQ ID NO: 5 - protein, 107 aa, synthetic construct
DIQMTQSPSSLSASVGDRVTITCLASEDIYDNLAWYQQKPGKAPKLLIYYASSLQDGVPSRFSGSGSGTDYTLTISSLQPEDFATYYCLQDSEYPWTFGGGTKVEIK

SEQ ID NO: 6 - protein, 12 aa, synthetic construct
GFSLTTYGMGVG

SEQ ID NO: 7 - protein, 16 aa, synthetic construct
NIWWDDDKYYNPSLKN

SEQ ID NO: 8 - protein, 16 aa, synthetic construct
IGPIKYPTAPYRYFDF

SEQ ID NO: 9 - protein, 11 aa, synthetic construct
LASEDIYDNLA

SEQ ID NO: 10 - protein, 7 aa, synthetic construct
YASSLQD

SEQ ID NO: 11 - protein, 9 aa, synthetic construct
LQDSEYPWT

SEQ ID NO: 12 - protein, 452 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 13 - protein, 446 aa, synthetic construct
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 213 aa, synthetic construct
DIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 446 aa, synthetic construct
QVQLVQSGAEVKKPGSSVKVSCKASGYTFTDNYMIWVRQAPGQGLEWMGDINPYNGGTTFNQKFKGRVTITADKSTSTAYMELSSLRSEDTAVYYCARESPYFSNLYVMDYWGQGTLVTVSSASTKGPSVFPLAPCSRSTSESTAALGCLVKDYFPEPVTVSWNSGALTSGVHTFPAVLQSSGLYSLSSWTVPSSSLGTKTYTCNVDHKPSNTKVDKRVESKYGPPCPPCPAPEFLGGPSVFLFPPKPKDTLMISRTPEVTCVWDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 217 aa, synthetic construct
EIVLTQSPATLSLSPGERATLSCKASQSVDYDGDNYMNWYQQKPGQAPRLLIYAASNLESGIPARFSGSGSGTDFTLTISSLEPEDFAVYYCHLSNEDLSTFGGGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 446 aa, synthetic construct
QDQLVESGGGWQPGRSLRLSCAASGFTFSSYGMHWVRQAPGEGLEWVAVIWYDGSNKYYADSVKGRFTISRDNSKNTLYLQMNSLRAEDTAVYYCARGDYEVDYGMDVWGQGTTVTVASASTKGPSVFPLAPSSKSTSGGTAALGCLVKDYFPEPVTVSWNSGALTSGVHTFPAVLQSSGLYSLSSWTVPSSSLGTQTYICNVNHKPSNTKVDKRVEPKSCDKTHTCPPCPAPELLGGPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 213 aa, synthetic construct
AIQLTQSPSSLSASVGDRVTITCRASQGISNALAWYQQKPGKAPKLLIYDASSLESGVPSRFSGSGSGTDFTLTISSLQPEDFATYYCQQFNSYPWTFGQGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 446 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 221 aa, synthetic construct
DIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKRTVAAPSVFIFPPSDEQLKSGTASVVVCLLNNFYPREAKVQWKVDNALQSGNSQESVTEQDSKDSTYSLSSTLTLSKADYEKHKVYACEVTHQGLSSPVTKSFNRGEC